(19) United States
(12) Patent Application Publication
(10) Pub. No.: US 2018/0125791 A1
(43) Pub. Date: May 10, 2018

(54) POLYMER NANOCAPSULES FOR PROTEIN DELIVERY

(71) Applicant: THE UNIVERSITY OF MASSACHUSETTS, Boston, MA (US)

(72) Inventors: Vincent M. Rotello, Amherst, MA (US); Ryan F. Landis, Amherst, MA (US); Moumita Ray, Amherst, MA (US)

(21) Appl. No.: 15/572,862

(22) PCT Filed: May 10, 2016
(86) PCT No.: PCT/US16/31582

§ 371 Date: Nov. 9, 2017

Related U.S. Application Data
(60) Provisional application No. 62/160,787, filed on May 13, 2015.

(51) Int. Cl. A61K 9/51; B01J 13/02; A61K 47/69; C08G 61/12; C08G 61/08

(52) U.S. Cl. CPC A61K 9/5146; B01J 13/02; A61K 47/6935; C08G 61/123; C08G 2261/418; C08G 2261/1424; C08G 2261/1432; C08G 2261/3324; C08G 2261/3342; C08G 61/08

(57) ABSTRACT

A nanocapsule includes a shell including a polymer and a protein, a nucleic acid, or a combination thereof. The polymer has repeating units of Formula (I) (I) wherein X, L1, R1, and y are as described herein. The nanocapsule further includes a core comprising an oil. A composition is also disclosed including a plurality of the nanocapsules dispersed in an aqueous solution. The nanocapsules prepared according to the methods described herein are particularly useful for the delivery of proteins and nucleic acids into cells.

### STATEMENT REGARDING FEDERALLY SPONSORED RESEARCH & DEVELOPMENT

This invention was made with government support under award number EB014277 awarded by the National Institutes of Health (NIH). The government has certain rights in the invention.

### BACKGROUND OF THE INVENTION

Protein transduction domains (PTDs) are oligo- or polycationic peptides that can facilitate cellular uptake of many different moieties, including small molecules, proteins, antibodies, DNA, RNA, nanoparticles, and the like. PTDs are primarily composed of cationic amino acid sequences including arginine and lysine residues. PTDs have been the subject of intensive research because it is well known that the cell membrane limits the transport of highly charged molecules. As highly cationic molecules, the ability of PTDs to readily cross the cell membrane is fundamentally important for gaining new insights into membrane transport. The ability of PTDs to deliver the aforementioned moieties into mammalian cells creates possibilities for new therapies and enhanced existing therapies.

Although the number of known PTDs has increased significantly, the design of synthetic analogs that capture the unique biological properties of PTDs remains a challenge. There is a significant need for new synthetic mimics of PTDs having improved cell-penetrating properties.

Accordingly, positively charged synthetic polymers as carriers for various therapeutic moieties have been the subject of intensive research and development. However, there remains a continuing need in the art for a stable, nano-sized, synthetic system suitable for efficiently delivering proteins into cells.

### BRIEF SUMMARY OF EMBODIMENTS OF THE INVENTION

One embodiment is a nanocapsule comprising a shell comprising a polymer and a protein, a nucleic acid, or a combination thereof, the polymer comprising repeating units of Formula (I)

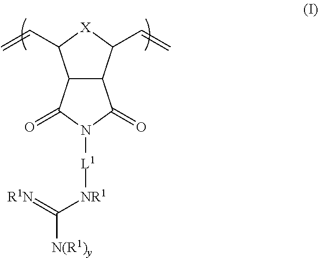

wherein X is independently at each occurrence —O—, —S—, —CH₂—, —(CR³R⁴)—, or

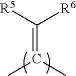

wherein R³ and R⁴ are independently at each occurrence a C₁₋₆ alkyl group and R⁵ and R⁶ are independently at each occurrence hydrogen or a C₁₋₆ alkyl group; L¹ is independently at each occurrence a divalent group that is (—CH₂-)_{z}, wherein is z is an integer from 1 to 10, a divalent a C₁₋₂₀ alkylene oxide group, or a divalent polyethylene oxide group; R¹ is independently at each occurrence hydrogen, a C₁₋₁₂ alkylene group, or a C₁₋₆—(C═O)O-alkylene group; y is 2 or 3; and a core defined by the shell, the core comprising an oil.

Another embodiment is a composition comprising a plurality of nanocapsules dispersed in an aqueous solution.

Another embodiment is a method of preparing the nanocapsule, the method comprising contacting a first aqueous solution comprising the copolymer with a second aqueous solution comprising the protein, nucleic acid, or combination thereof, to provide a reaction mixture comprising a polymer-protein complex, a polymer-nucleic acid complex or a combination thereof; and contacting the reaction mixture with an emulsion comprising spherical droplets of the oil dispersed in an aqueous phase to provide the nanocapsule.

Another embodiment is a method of delivering a protein, a nucleic acid, or a combination thereof into a cell, the method comprising contacting the nanocapsule with a cell.

These and other embodiments are described in detail below.

### BRIEF DESCRIPTION OF THE DRAWINGS

FIG. 1 is a chemical scheme showing a synthetic route to Compound 1.

FIG. 2 is a chemical scheme showing a synthetic route to Compound 2.

FIG. 3 is a chemical scheme showing a synthetic route to Compound 5.

FIG. 4 is a chemical scheme showing a synthetic route to Compound 7.

FIG. 5 is a chemical scheme showing a synthetic route to Polymer 9.

FIG. 6(A) is a schematic illustration showing the preparation of the nanocapsules. FIG. 6(B) is a schematic illustration showing the preparation of the nanocapsules.

FIG. 7 shows zeta potential measurements for the nanocapsules.

FIG. 8 shows transmission electron micrographs of the nanocapsules (scale bar is 100 nanometers).

FIG. 9 shows a digital photograph of a solution of the nanocapsules encapsulating Nile Red dye (left), and the same solution after centrifugation (right).

FIG. 10 shows HeLa cell viability after a 24 hour incubation with the polymer nanocapsules at varying concentrations.

FIG. 11 shows confocal microscopy of HeLa cells after incubation with nanocapsules including (A, B) green fluorescent protein (GFP) having a negatively charged tag comprising a peptide having ten units of glutamic acid (“E-10”), (C, D) red fluorescent protein (DsRed), and (E) Factor inhibiting Hypoxia-inducible factor (HIF)-1 (“FIH”) protein tagged with fluorescein isothiocyanate (FIH-FITC). The light areas are indicative of fluorescence. Also shown are the corresponding bright field microscopy images of the HeLa cells after incubation with the polymer nanocapsules containing E-10 tagged GFP (F, G), DsRed (H, I), and FIH-FITC (J) proteins.

FIG. 12 shows confocal microscopy of HeLa cells after incubation with Nile Red-loaded nanocapsules (A). The light areas are indicative of fluorescence arising from the Nile Red. Also shown are the corresponding bright field microscopy images of the HeLa cells after incubation with the polymer nanocapsules containing Nile Red (B).

FIG. 13 shows confocal microscopy of HeLa cells after incubation with nanocapsules including siRNA (A). The light areas are indicative of fluorescence arising from the Cyanine dye used to label the siRNA. Also shown are the corresponding bright field microscopy images of the HeLa cells after incubation with the polymer nanocapsules containing siRNA (B).

FIG. 14 shows confocal microscopy of HeLa cells after incubation with nanocapsules including unmodified GFP (A). The light areas are indicative of fluorescence arising from the GFP. Also shown are the corresponding bright field microscopy images of the HeLa cells after incubation with the polymer nanocapsules containing unmodified GFP (B).

FIG. 15 shows confocal microscopy images of the intestinal stem cells of fruit flies that have been orally administered polymer nanocapsules. FIG. 15(A) shows the intestinal stem cells of fruit flies that were fed sucrose (control); FIG. 15(B) shows the intestinal stem cells of fruit flies that were fed GFP (control); FIG. 15(C) shows the intestinal stem cells of fruit flies that were fed the GFP-loaded polymer nanocapsules. The light areas are indicative of fluorescence arising from GFP.

### DETAILED DESCRIPTION OF THE INVENTION

The present inventors have prepared polymer nanocapsules from guanidinium-containing polymers. The inventors have advantageously discovered that the spontaneous assembly of the polymers, one or more proteins, nucleic acids, or a combination thereof, and an oil (e.g., an oil-in-water emulsion) yields stable nanocapsules. The polymers can be prepared by ring-opening metathesis polymerization (ROMP) of suitable monomers. The polymer nanocapsules are particularly useful for delivering proteins into cells.

One aspect of the present disclosure is a nanocapsule. The nanocapsules can be core-shell nanocapsules, comprising a shell and a core defined by the shell. The shell comprises a polymer and a protein, a nucleic acid, or a combination thereof.

The polymer comprises repeating units of Formula (I)

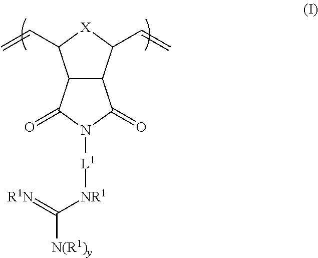

wherein X is independently at each occurrence —O—, —CH₂—, —(CR³R⁴)—, or

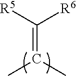

wherein R³ and R⁴ are independently at each occurrence a substituted or unsubstituted C₁₋₆ alkyl group and R⁵ and R⁶ are independently at each occurrence hydrogen or a substituted or unsubstituted C₁₋₆ alkyl group; L¹ is independently at each occurrence a divalent group that is (—CH₂-)_{y}, wherein is y is an integer from 1 to 10, a divalent substituted or unsubstituted C₁₋₂₀ alkylene oxide group, or a divalent polyethylene oxide group; R¹ is independently at each occurrence hydrogen, a substituted or unsubstituted C₁₋₁₂ alkylene group, or a substituted or unsubstituted C₁₋₆—(C═O)O-alkylene group; and y is 2 or 3. In some embodiments, R⁵ and R⁶ are each independently propylene (i.e., a C₃ alkyl group). In some embodiments, X is —O—. In some embodiments, L¹ is propylene. In some embodiments, R¹ is hydrogen. In some embodiments, R¹ is hydrogen and y is 3. When y is 3, the repeating unit of Formula (I) comprises a positively charged group, for example a guanidinium group. In some embodiments, when the repeating unit of Formula (I) comprises the positively charged guanidinium group, the copolymer can further comprise a counterion. The counterion can be, for example, chloride, bromide, trifluoroacetate, acetate, citrate, lactate, succinate, propionate, butyrate, ascorbate, maleate, folate, iodide, fluoride, phosphate, sulfonate, carbonate, or a combination thereof. In a specific embodiment, each occurrence of X is —O—; each occurrence of L¹ is propylene; each occurrence of R¹ is hydrogen; and y is 3. In some embodiments, the polymer can comprise 10 to 100 repeating units according to Formula (I).

In some embodiments, the polymer is a copolymer further comprising repeating units of Formula (II)

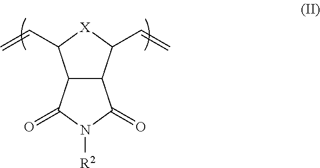

wherein X is independently at each occurrence —O—, —S—, —CH₂—, —(CR³R⁴)—, or

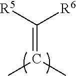

wherein R³ and R⁴ are independently at each occurrence a substituted or unsubstituted C₁₋₆ alkyl group and R⁵ and R⁶ are independently at each occurrence hydrogen or a substituted or unsubstituted C₁₋₆ alkyl group; and R² is independently at each occurrence a substituted or unsubstituted C₁₋₁₂ alkylene group, a substituted or unsubstituted C₆₋₂₀ arylene group, a substituted or unsubstituted C₁₋₂₀ alkylene oxide group, a polyethylene oxide group, or a zwitterionic group. A polyethylene oxide group can be a group of the formula —(CH₂CH₂O)ₙ—R⁷, wherein n is 10 to 1000, and R⁷ is hydrogen or a C₁₋₆ alkyl group, preferably a methyl group. A zwitterionic group is a group of the formula -L₂-A-B—C, wherein L₂ is a linking group that is (—CH₂-)ₚ, wherein is p is an integer from 1 to 10, A is a center of permanent positive charge or a center of permanent negative charge, B is a divalent group comprising a C₁₋₁₂ alkylene group, a C₆₋₃₀ arylene group, or an alkylene oxide group, and C is a center of permanent negative charge or a center of permanent positive charge, provided that the zwitterion has an overall net charge of zero (i.e., the zwitterion is net neutral). For example, in an embodiment wherein A is a center of permanent positive charge, C is a center of permanent negative charge. For example, in an embodiment wherein A is a center of permanent negative charge, C is a center of permanent positive charge. In some embodiments, a center of permanent positive charge can include a quaternary ammonium group, a phosphonium group, a sulfonium group, and the like. In some embodiments, the center of permanent positive charge is preferably an ammonium group. In some embodiments, a center of permanent negative charge can include a sulfonate group, a phosphonate group, a carboxylate group, a thiolate group, and the like. In some embodiments, the zwitterionic group is a sulfobetaine group or a carboxy betaine group. In an embodiment, the zwitterionic group is a sulfobetaine group wherein L₂ is ethylene, A is ammonium (e.g., a dimethyl ammonium), B is propylene, and C is a sulfonate group. In an embodiment, the zwitterionic group is a carboxy betaine group wherein L₂ is ethylene, A is ammonium (e.g., a dimethyl ammonium), B is methylene, and C is a carboxylate group. In some embodiments, R² is preferably a C₁₋₂₀ alkylene oxide group. For example, R² can be a group having the structure

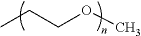

wherein n is 1, 2, 3, or 4. In some embodiments, n is 4.

In a specific embodiment, the polymer is a copolymer comprising repeating units of Formula (I) and (II), wherein each occurrence of X is —O—; each occurrence of L¹ is propylene; each occurrence of R¹ is hydrogen; y is 3; and each occurrence of R² is a group having the structure

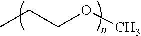

wherein n is 4.

In a specific embodiment, the copolymer is of the Formula (III)

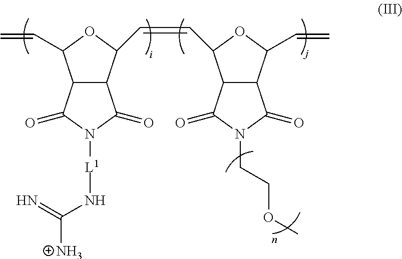

wherein L¹ is independently at each occurrence a divalent group that is (—CH2-)y, wherein is y is an integer from 1 to 10, preferably wherein L¹ is a propylene group; n is 1, 2, 3, or 4, preferably 4; and i and j are each integers greater than 1, for example 2 to 50.

The copolymer can be a block copolymer or a random copolymer. In some embodiments, the copolymer is a random copolymer. In some embodiments, the molar ratio of units of Formula (I) to units of Formula (II) is 1:10 to 10:1, for example 1:5 to 5:1, for example 1:2 to 2:1, for example 1:1. Stated another way, in some embodiments, the ratio of i to j of Formula (III) can be 1:10 to 10:1, for example 1:5 to 5:1, for example 1:2 to 2:1, for example 1:1.

In some embodiments, the polymer has a number average molecular weight, as determined by gel permeation chromatography, of 1,000 to 100,000 grams per mole (g/mole), for example 10,000 to 100,000 g/mole, for example 10,000 to 75,000 g/mole, for example 10,000 to 50,000 g/mole, for example 10,000 to 30,000 g/mole.

The polymer can be prepared by any method which is generally known. For example, the polymer can be prepared by ring opening metathesis polymerization (ROMP) of a suitable cyclic olefin monomer (e.g., norbornene, oxanorbornene, derivatives thereof, and the like) in the presence of in the presence of a ROMP catalyst such as a ruthenium-containing catalyst. An example of such a procedure is described in the working examples below.

In addition to the polymer, the shell of the nanocapsule also comprises a protein, a nucleic acid, or a combination thereof. In some embodiments, the shell comprises a protein. The term “protein” as used herein, refers to a plurality of amino acid residues (e.g., generally greater than 10) joined together by peptide bonds, and having a molecular weight greater than 500 Daltons (Da), preferably greater than 5,000 Da. For example, the protein can have a molecular weight of 500 Da to 200,000 Da, preferably 5,000 Da to 200,000 Da, more preferably 20,000 to 200,000 Da. This term is also intended to include fragments, analogues and derivatives of a protein wherein the fragment, analogue or derivative retains essentially the same biological activity or function as a reference protein. The protein can be a linear structure or a non-linear structure having a folded, for example tertiary or quaternary, conformation. The protein can have one or more prosthetic groups conjugated to it, for example the protein may be a glycoprotein, lipoprotein or chromoprotein. Proteins useful for making the nanocapsules can have a variety of functions, and thus can include, but are not limited to structural protein, non-structural protein, coat protein, capsid protein, core protein, envelope protein, matrix protein, transmembrane protein, membrane associated protein, non-structural protein, nucleocapsid protein, filamentous protein, capping protein, crosslinking protein, glycoprotein, and motor protein. Preferably, the protein is a biologically active protein, for example, the protein can comprise glycoproteins, serum albumins and other blood proteins, hormones, enzymes, receptors, antibodies, interleukins, interferons, and the like, and combinations thereof.

In some embodiments, the protein has a negative surface charge capable of interacting with the above-described positively charged copolymer. Thus, in some embodiments, the protein and the copolymer are present in the nanocapsule in the form of a polymer-protein complex, wherein the complexation is facilitated by non-covalent interactions, for example, electrostatic interactions, hydrogen bonding interactions, van der waals interactions, cation-pi interactions, or a combination thereof. In some embodiments, the protein and the copolymer are preferably not covalently bonded (i.e., no covalent bonds exist between the protein and the copolymer). In some embodiments, the polymer-protein complex can comprise at least 1, 2, 3, 4, 5, 10, or more proteins per polymer chain. In an embodiment, the polymer-protein complex comprises no more than 10 proteins per polymer chain. In some embodiments, the overall net charge of the protein-polymer complex is positive.

In some embodiments, the protein can include a negatively charged group conjugated to the protein. Without wishing to be bound by theory, it is believed that a negatively charged group can facilitate formation of the polymer-protein complex. In some embodiments, the negatively charged group can be conjugated to a terminus of the protein (i.e., the C terminus, the N terminus, or both), or to a surface-available site of the protein. For example, the negatively charged group can include a carboxylate group, a sulfonate group, a phosphonate group, or a combination thereof. In some embodiments, the negatively charged group can be a group that is negatively charged at physiological pH, for example a peptide or an amino acid group comprising one or more residues of glutamic acid, aspartic acid, or a combination thereof. In some embodiments, the protein comprises a targeting group. As used herein, the term “targeting group” refers to any substance that binds to a component associated with a cell, tissue, or organ. For example, a targeting group can be a polypeptide, glycoprotein, nucleic acid, small molecule, carbohydrate, lipid, an antibody, a receptor, a nucleic acid targeting agent (e.g. an aptamer) that binds to a cell type specific marker, and the like. In some embodiments, the targeting group can be a nuclear targeting group, for example the targeting group can have a nuclear localization signal (NLS). For example, the targeting group can be SV40 Large T-Antigen, c-Myc, and EGL-13, each of which contain NLS amino acid sequences that can target a cell nucleus. In general, the NLSs can be attached at the C- or N-terminus of the proteins. The SV40 Large T-Antigen has the NLS sequence PKKKRKV; C-Myc has the NLS sequence PAAKRVKLD; and EGL-13 has the NLS sequence MSRRRKANPTKLSENAKKLAKEVEN, wherein the letters of the aforementioned sequences represent the corresponding amino acid. The negatively charged group, the targeting group, and any other group that can be included in the protein can be naturally present on the surface of the protein, or can be incorporated on the surface of the protein using any synthetic coupling method that is generally known for conjugation of such groups to proteins.

In some embodiments, the protein is a therapeutic protein. As used herein, the term “therapeutic protein” refers to a protein, peptide, or the like, which provides a therapeutic effect. The term “protein” can include oligopeptides, proteins, recombinant proteins, and conjugates thereof, particularly those identified as having therapeutic potential. The proteins can be naturally occurring or synthetic (e.g., engineered). Proteins and peptides conjugated to non-protein compounds, for example non-protein therapeutic compounds are also included in the scope of the terms.

In some embodiments, the protein is a therapeutic protein comprising factor VIII, b-domain deleted factor VIII, factor VIIa, factor IX, anticoagulants; hirudin, alteplase, tpa, reteplase, tpa, tpa-3 of 5 domains deleted, insulin, insulin lispro, insulin aspart, insulin glargine, long-acting insulin analogs, hgh, glucagons, tsh, follitropin-beta, fsh, gm-csf, pdgh, ifn alpha2, ifn alpha2a, ifn alpha2b, inf-aphal, consensus ifn, ifn-beta, ifn-beta 1b, ifn-beta 1a, ifn-gamma (e.g., 1 and 2), ifn-lambda, ifn-delta, it-2, it-11, hbsag, ospa, murine mab directed against t-lymphocyte antigen, murine mab directed against tag-72, tumor-associated glycoprotein, fab fragments derived from chimeric mab directed against platelet surface receptor gpII(b)/III(a), murine mab fragment directed against tumor-associated antigen ca125, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, human epidermal growth factor receptor 2, human mab directed against cytokeratin tumor-associated antigen anti-ctla4, chimeric mab directed against cd 20 surface antigen of b lymphocytes dornase-alpha dnase, beta glucocerebrosidase, tnf-alpha, il-2-diptheria toxin fusion protein, tnfr-lgg fragment fusion protein laronidase, dnaases, alefacept, darbepoetin alpha (colony stimulating factor), tositumomab, murine mab, alemtuzumab, rasburicase, agalsidase beta, teriparatide, parathyroid hormone derivatives, adalimumab (lgg1) anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker, lbritumomab tiuxetan, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, etanercept, antidiuretic hormones, prolactin, thyroid stimulating hormone, a clustered regularly interspaced short palindromic repeat (CRISPR) associated protein, a caspase protein, a tyrosine recombinase enzyme, a ribonuclease, and combinations thereof.

In some embodiments, the protein comprises a clustered regularly interspaced short palindromic repeat (CRISPR) associated protein, a caspase protein (e.g., Caspase 3), a tyrosine recombinase enzyme, or a ribonuclease. In an embodiment, the protein comprises a CRISPR. CRISPRs are essential components of a recently discovered, nucleic-acid-based adaptive immune system that is widespread in bacteria and archaea and serves as protection against phages and other invading nucleic acids. The protein can be a CRISPR-associated protein, for example CRISPR-associated protein 9 (Cas9).

In some embodiments, the shell comprises a nucleic acid, for example a ribonucleic acid (RNA). The term “nucleic acid” or “polynucleotide” includes DNA molecules and RNA molecules. A nucleic acid may be single-stranded or double-stranded. Nucleic acids can contain known nucleotide analogs or modified backbone residues or linkages, which are synthetic, naturally occurring, and non-naturally occurring, which have similar binding properties as the reference nucleic acid. Examples of such analogs include, without limitation, phosphorothioates, phosphoramidates, methyl phosphonates, chiral-methyl phosphonates, 2-O-methyl ribonucleotides, peptide-nucleic acids (PNAs). A nucleic acid can be obtained by a suitable method known in the art, including isolation from natural sources, chemical synthesis, or enzymatic synthesis. Nucleotides may be referred to by their commonly accepted single-letter codes. The nucleic acids can further include conjugated variants thereof, where the conjugated group can be another nucleic acid, or a molecule including a lipid, a peptide, a dye, and the like, or a combination thereof. Suitable nucleic acids can have variable chain lengths. In some embodiments, the nucleic acid can comprise at least 2 nucleotides, for example 5 to 10,000 nucleotides. Within this range, the nucleic acid can have at least 10, or at least 100, or at least 500, or at least 1,000 nucleotides. Also within this range, the nucleic acid can have less than or equal to 8,000 nucleotides, or less than or equal to 5,000 nucleotides, or less than or equal to 2,500 nucleotides, or less than or equal to 1,500 nucleotides, or less than or equal to 1,000 nucleotides, or less than or equal to 500 nucleotides, or less than or equal to 100 nucleotides. RNA can include, for example, small interfering RNAs (siRNAs), microRNAs (miRNAs), small hairpin RNAs (shRNAs), and others, or a combination thereof. In an embodiment, the RNA is siRNA. In an embodiment, the RNA is siRNA. In some embodiments, the nucleic acid has a net negative charge capable of interacting with the above-described positively charged copolymer. Thus, in some embodiments, the nucleic acid and the copolymer are present in the nanocapsule in the form of a polymer-nucleic acid complex, wherein the complexation is facilitated by non-covalent interactions, for example, electrostatic interactions, hydrogen bonding interactions, van der waals interactions, or a combination thereof. In some embodiments, the nucleic acid and the copolymer are preferably not covalently bonded (i.e., no covalent bonds exist between the nucleic acid and the copolymer). In some embodiments, the polymer-nucleic acid complex can comprise at least 1, 2, 3, 4, 5, 10, or more nucleic acids per polymer chain. In an embodiment, the polymer-nucleic acid complex comprises no more than 10 nucleic acids per polymer chain. In some embodiments, the overall net charge of the protein-nucleic acid complex is positive.

In addition to the shell, the nanocapsules of the present disclosure further comprise a core comprising an oil. In some embodiments, the oil can include a silicone oil, a hydrocarbon oil, a petroleum oil, a fuel oil, a wax, a fatty acid (e.g., a C₁₂₋₂₄ fatty acid), a liquid lipid, a fluorinated oil, a non-volatile oil, a volatile oil, an aromatic oil, an oil derived from a plant material, an oil derived from an animal material, an oil derived from a natural source, a distilled oil, an extracted oil, a cooking oil, a vegetable oil, a food oil, a lubricant, a reactive material that is predominantly hydrocarbon in composition, an epoxy material, an adhesive material, a polymerizable material, a thermotropic liquid crystal, a lyotropic liquid crystal, an acidic oil, a basic oil, a neutral oil, a natural oil, a polymer oil, a synthetic oil, or a combination thereof. In some embodiments, the oil comprises a liquid lipid comprising soybean oil, sunflower oil, corn oil, olive oil, palm oil, cottonseed oil, colza oil, peanut oil, coconut oil, castor oil, linseed oil, borage oil, evening primrose oil, marine oils (e.g., fish oils and algae oils), oils derived from petroleum (e.g., mineral oil, liquid paraffin and vaseline), short-chain fatty alcohols, medium-chain aliphatic branched fatty alcohols, fatty acid esters with short-chain alcohols (e.g., isopropyl myristate, isopropyl palmitate, isopropyl stearate, dibutyl adipate), medium-chain triglycerides (MCT) (e.g., capric and caprylic triglycerides and other oils in the Miglyol® series), C₁₂-C₁₆octanoates, fatty alcohol ethers (e.g., dioctyl ether), and combinations thereof. In a specific embodiment, the oil comprises a C₁₂₋₂₄ fatty acid, for example the oil can be linoleic acid, oleic acid, myristoleic acid, palmitoleic acid, arachidonic acid, eicosapentaenoic acid, docosahexaenoic acid, or a combination thereof. The fatty acid can be a saturated fatty acid or an unsaturated fatty acid. In some embodiments, the oil is selected such that the surface of the oil core comprises a negative charge, for example due to the presence of a carboxylate group. Without wishing to be bound by theory, it is believed that the negative charge on the surface of the oil core facilitates electrostatic interactions between the positively-charged polymer, the positively-charged polymer-protein complex, the positively charged polymer-nucleic acid complex, or a combination thereof, which can lead to the spontaneous formation of the nanocapsules.

In some embodiments, the oil preferably comprises linoleic acid. In some embodiments the oil comprises linoleic acid and decanoic acid. In some embodiments, the linoleic acid and the decanoic acid can be present in a molar ratio of 1:1.

In some embodiments, the nanocapsule can optionally further comprise an additional active ingredient (i.e., an active ingredient different from the protein and the nucleic acid). For example, the nanocapsule can optionally further comprise a peptide, a nucleic acid, an oligonucleotide, a polynucleotide, a hydrophobic drug, an imaging agent, or a combination thereof. In some embodiments, the nanocapsules can further comprise a nucleic acid, such as an oligonucleotide, interference RNA, guide RNA (gRNA), a DNA plasmid or a polynucleotide, preferably gRNA. As used herein, a “hydrophobic drug,” is a water insoluble drug. A “drug” is a therapeutically active substance which is delivered to a living subject to produce a desired effect, such as to treat a condition of the subject. A “water insoluble drug” can have a solubility of less than 0.1 mg/mL in distilled water at 250° C. Hydrophobic drugs can include but are not limited to amphotericin, anthralin, beclomethasone, betamethasone, camptothecin, curcumin, irinotecan, topotecan, dexamethasone, paclitaxel, doxorubicin, docetaxel, and the like, or a combination thereof. The additional active ingredient can be present in the shell of the nanocapsule, the core of the nanocapsule, or both. In some embodiments, the additional active ingredient can be present in the nanocapsule in an amount of 0 to 50 weight percent (wt. %) based on the total weight of the nanocapsule, for example 1 to 50 wt. %. Within this range, the active ingredient can be present in an amount of greater than or equal to 2, 5, 10, or 25 wt. %. Also within this range, the active ingredient can be present in an amount of less than or equal to 40, 30, 20, or 10 wt. %.

In some embodiments, the nanocapsules have an average diameter of less than or equal to 100 nanometers (nm), for example 1 to 100 nm, or 5 to 100 nm, or 10 to 100 nm, or 15 to 100 nm, or 15 to 90 nm, or 15 to 85 nm, or 20 to 80 nm, or 20 to 75 nm. In some embodiments, the overall net charge of the surface of the nanocapsule is positive. For example, in some embodiments, the surface of the nanocapsule can have a charge of 1 to 10 millivolts (mV), or 1 to 5 mV. The net positive surface charge of the nanocapsules of the present disclosure is believed to be important for obtaining an interaction between the nanocapsule and a biological surface, particularly between the nanocapsule and a cell membrane.

Another aspect of the present disclosure includes a composition comprising a plurality of nanocapsules. As used herein, “plurality of nanocapsules” refers to a composition comprising more than 1 nanocapsule, for example more than 10 nanocapsules. The nanocapsules can include nanocapsules having the above-described structure and components. The composition comprises the plurality of nanocapsules dispersed in an aqueous solution. The aqueous solution can comprise water, deionized water, a buffer (e.g., phosphate buffered saline, phosphate buffer, and the like), and the like, or a combination thereof. In some embodiments, the composition comprises 1 to 50 volume percent (vol. %) of the nanocapsules, for example 1 to 20 vol. %, for example 5 to 15 vol. %, based on the total volume of the composition. Accordingly, in some embodiments, the composition comprises 50 to 99 vol. %, or 80 to 99 vol. %, or 85 to 95 vol. % of the aqueous solution, based on the total volume of the composition.

Another aspect of the present disclosure is a method of preparing the above-described nanocapsule. The method of preparing the nanocapsule comprises contacting a first aqueous solution comprising the copolymer with a second aqueous solution comprising the protein, nucleic acid, or combination thereof, to provide a reaction mixture comprising a polymer-protein complex, a polymer-nucleic acid complex, or a combination thereof. The first and second aqueous solutions can each independently comprise water, deionized water, a buffer (e.g., phosphate buffered saline, phosphate buffer, and the like), and the like, or a combination thereof. In some embodiments, the contacting is carried out under conditions effective to provide the polymer-protein complex or polymer-nucleic acid complex, for example at a pH of 5 to 13, at a temperature of 18 to 28° C., and for 1 minute to 1 hour, or 1 to 30 minutes, or 1 to 15 minutes.

The method further comprises contacting the reaction mixture with an emulsion comprising droplets (e.g., spherical droplets) comprising the oil dispersed in an aqueous phase to provide the nanocapsules. In some embodiments, the nanocapsules are provided as an aqueous dispersion. The aqueous phase can be the same or different as the first and/or the second aqueous solutions as described above. The emulsion can be prepared by any method that is generally known. For example, the emulsion can be prepared by adding the oil to the aqueous phase, and subsequently agitating the mixture (e.g., by shaking). In some embodiments, the emulsion can comprise 0.1 to 10 vol. %, or 0.1 to 1 vol. %, or 0.1 to 0.5 vol. % of the oil and 90 to 99.9 vol. %, or 99 to 99.9 vol. %, or 99.5 to 99.9 vol. % of the aqueous phase. As described above, the oil can be selected such that the surface of the oil droplet comprises a negative charge. Accordingly, without wishing to be bound by theory, contacting the reaction mixture comprising the polymer-protein complex or the polymer-nucleic acid complex with the emulsion can result in the spontaneous diffusion of the complex, the copolymer, or both, to the surface of the oil droplet due to electrostatic interactions, providing the resulting nanocapsule with stability. Thus, there is no need for creating covalent bonds between the components of the nanocapsules prepared according to the methods disclosed herein. In some embodiments, the nanocapsules prepared according the above-described method are stable for at least 3 days in aqueous solution at a temperature of 25° C., for example, an aqueous solution have a pH of 6 to 8.

The method of preparing the nanocapsules of the present disclosure is further described in detail in the working examples below.

Another aspect of the present disclosure is a method of delivering a protein, a nucleic acid, or a combination thereof to a cell. The method comprises contacting the above-described nanocapsule with a cell. In some embodiments, the method results in delivery of the protein or nucleic acid to the nucleus of the cell. In some embodiments, the cell can be a mammalian cell, for example, a human cell. In some embodiments, the cell can be a neuronal cell, a T-cell, a fibroblast, an epithelial cell, a tumor cell, a muscle cell, a skin cell, or an immune system cell. In some embodiments, the protein or nucleic acid is released from the nanocapsule upon contacting the nanocapsule with the cell. In some embodiments, the nanocapsule is transported across a cell membrane upon contact with the cell. In some embodiments, the protein or nucleic acid is released from the nanocapsule after being transported across a cell membrane. In some embodiments, at least 50% of the protein or nucleic acid is released from the nanocapsule, for example, at least 60%, or at least 75%, or at least 90%, or at least 95%, or at least 99%, based on the total amount of the protein or nucleic acid present in the nanocapsule.

In some embodiments, the contacting is performed in vitro. In some embodiments, the contacting is performed in vivo, for example, in the body of a subject or patient, for example, a human or other animal. In an embodiment, the nanocapsule is present in the cell in an amount effective to provide a detectable effect in the subject during or after release of the protein, nucleic acid, or both, e.g., a therapeutic effect. In some embodiments, the observed or detectable effect arises from cell penetration of the nanocapsule and release of the protein from the nanocapsule.

The invention is further illustrated by the following non-limiting examples.

### EXAMPLES

The monomers and polymers used to prepare the guanidinium-containing polymer nanocapsules of the present disclosure were prepared according to the following procedures.

Synthesis of Compound 1.

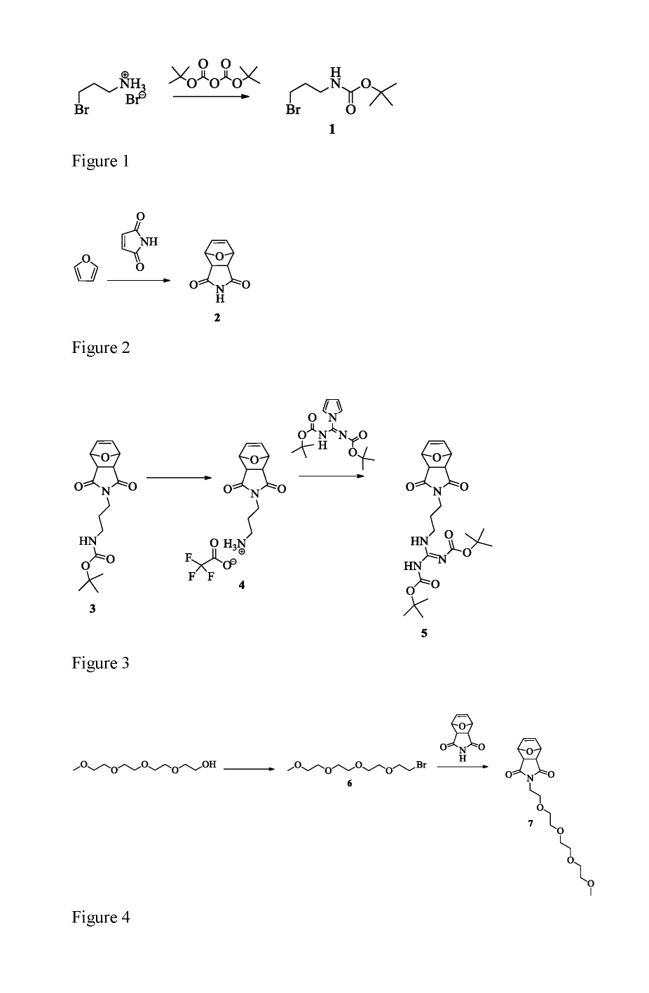

Compound 1 was prepared as summarized in the chemical scheme of FIG. 1. To a 500 milliliter (mL) round bottom flask equipped with a stir bar was added 150 mL of dichloromethane (DCM). 3-Bromopropylamine hydrobromide (10.0 grams, 45.7 millimoles) was added to the DCM solution. Triethylamine (Et₃N) (25.5 milliliters, 182.7 millimoles) was added to the reaction mixture. Di-tert-butyl dicarbonate (12.6 milliliters, 54.8 millimoles) was then added dropwise. After addition of di-tert-butyl dicarbonate, the reaction was stirred overnight at room temperature. The reaction mixture was concentrated by rotary evaporation, and diluted with 100 mL of diethyl ether, and extracted with 1 molar hydrochloric acid (HCl) (1×20 mL), saturated sodium bicarbonate (2×20 mL), and brine (1×20 mL). The organic layer was dried with sodium sulfate, filtered, and concentrated by rotary evaporation to yield compound 1 as a colorless liquid. Compound 1 was purified using column chromatography over silica gel. Compound 1 was characterized by proton nuclear magnetic resonance (′H NMR) spectroscopy. ¹H NMR (400 MHz, CDCl₃) 4.6 (br, 1H) 3.43 (t, 2H), 3.26 (br, 2H), 2.04 (t, 2H), 1.43 (s, 9H).

Synthesis of Compound 2.

Compound 2 was prepared as summarized in the chemical scheme of FIG. 2. In a pressure tube, furan (4.5 mL, 61.7 millimoles), maleimide (4.0 grams, 41.1 millimoles), and diethyl ether (5 mL) were added. The tube was sealed and heated at 100° C. overnight. The pressure tube was then cooled to room temperature. The precipitated solid was removed by filtration and washed with copious amounts of diethyl ether to isolate Compound 2 as a white solid. Compound 2 was used without further purification. ¹H NMR (400 MHz, MeOD) 11.14 (s, 1H), 6.52 (s, 2H), 5.12 (s, 2H), 2.85 (s, 2H).

Synthesis of Compound 5.

Compound 5 was prepared as summarized in the chemical scheme of FIG. 3. To a 100 mL round bottom flask equipped with a stir bar, 30 mL of dimethylformamide (DMF) was added. Compound 2 (2.36 grams, 14.3 millimoles) and potassium carbonate (7.9 grams, 57.2 millimoles) were added to the DMF. The reaction mixture was heated at 50° C. for five minutes. Potassium iodide (0.05 grams, 0.30 millimoles) and Compound 1 (3.47 grams, 14.6 millimoles) were then added to the reaction mixture and stirred at 50° C. overnight. The reaction mixture was cooled to room temperature, diluted to a volume of 150 mL with ethyl acetate and washed with water (7×50 mL) and brine (1×50 mL). The organic layer was dried with sodium sulfate, filtered, and concentrated by rotary evaporation to yield Compound 3 as a white solid. Compound 3 was purified using column chromatography over silica gel. ¹H NMR (400 MHz, CDCl₃) 6.51 (s, 2H), 5.26 (s, 2H), 5.03 (br, 1H), 3.56 (t, 2H), 3.05 (q, 2H), 2.86 (s, 2H), 1.73 (q, 2H) 1.45 (s, 9H).

To a 50 mL round bottom flask equipped with a stir bar, Compound 3 (2.0 grams, 6.2 millimoles) was added. Nitrogen gas was bubbled through DCM for five minutes, then 5 mL was added to the flask containing Compound 3. Trifluoroacetic acid (TFA, 5 mL) was added and the reaction mixture was stirred for two hours. Excess TFA was removed by rotary evaporation with DCM (3×) yielding Compound 4. Compound 4 was isolated as a white solid by washing with diethyl ether (3×10 mL), and used without further purification.

To a 100 mL round bottom flask equipped with a stir bar Compound 4 (1.2 grams, 3.6 millimoles), 45 mL acetonitrile (MeCN), and 5 mL of water were added. Triethylamine (4.7 mL, 33.5 millimoles) was added, followed by N,N′-Di-Boc-1H-pyrazole-1-carboxamidine (1.7 grams, 5.5 millimoles) in small portions. The reaction mixture was stirred at room temperature overnight. The reaction mixture was then diluted with 100 mL of ethyl acetate and extracted with water (2×50 mL) and brine (2×50 mL). The organic layer was dried with sodium sulfate, filtered, and concentrated by rotary evaporation to yield Compound 5. Compound 5 was purified using column chromatography over silica gel to yield a white solid. ¹H NMR (400 MHz, CDCL₃) 8.49 (t, 1H), 6.49 (s, 2H), 5.25 (s, 2H), 3.53 (t, 2H), 3.47 (q, 2H), 2.83 (s, 2H), 1.82 (quint, 2H), 1.49 (s, 18H).

Synthesis of Compound 7.

Compound 7 was prepared as summarized in the chemical scheme of FIG. 4. To a 250 mL round bottom flask tetraethylene glycol monomethyl ether (4.2 mL, 20.9 millimoles) and 80 mL of acetonitrile was added. The mixture was cooled to 0° C. and tetrabromomethane (8.4 grams, 25.1 millimoles) was added. Triphenylphosphine (6.6 grams, 25.3 millimoles) was then added in portions, and the reaction mixture was stirred for five minutes at 0° C. The reaction was warmed to room temperature and stirred overnight. The reaction mixture was concentrated by rotary evaporation and purified using column chromatography over silica gel to yield Compound 6 as a colorless oil. ¹H NMR (400 MHz, CDCL₃) 3.75 (t, 2H), 3.6 (br, 10H), 3.49 (t, 2H), 3.41 (t, 2H), 3.32 (s, 3H).

To a 100 mL round bottom flask equipped with a stir bar 30 mL of DMF was added. Compound 2 (2.84 grams, 17.2 millimoles) was added with potassium carbonate (9.48 grams, 68.7 millimoles). The reaction mixture was heated at 50° C. for five minutes. Potassium iodide (0.05 grams, 0.30 millimoles) and Compound 6 (4.9 grams, 18.0 millimoles) were added, and the reaction mixture was stirred at 50° C. overnight. The reaction mixture was then cooled to room temperature, diluted to 150 mL with ethyl acetate, and washed with water (7×50 mL) and brine (1×50 mL). The organic layer was dried with sodium sulfate, filtered, and concentrated by rotary evaporation to yield Compound 7. Compound 7 was isolated as a colorless oil following purification by column chromatography over silica gel. ¹H NMR (400 MHz, CDCL₃) 6.49 (s, 2H), 5.23 (s, 2H), 3.66 (t, 2H), 3.6 (br, 8H), 3.58 (br, 4H), 3.51 (t, 2H), 3.35 (s, 3H), 2.83 (s, 2H).

Synthesis of Polymer 9.

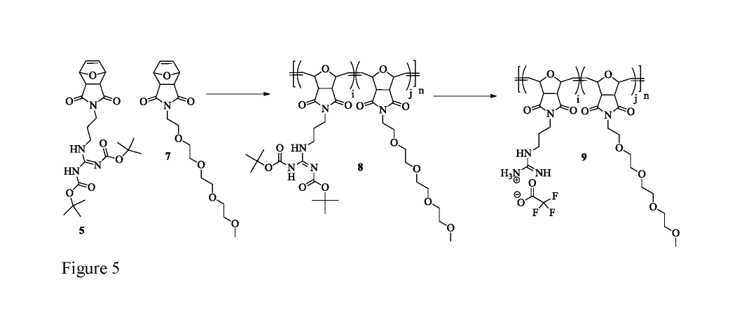

Polymer 9 was prepared as summarized in the chemical scheme of FIG. 5. To a 10 mL pear-shaped flask equipped with a stir bar Compound 5 (200 milligrams, 0.43 millimoles) and Compound 7 (153 milligrams, 0.43 millimoles) were added. In a separate vessel, DCM was purged with nitrogen gas for five minutes. Subsequently, 5 mL of the purged DCM was added to the flask containing Compounds 5 and 7. The reaction mixture was properly sealed with a septum and purged with nitrogen gas for two minutes. The nitrogen pressure was reduced to a steady stream. Hoveyda-Grubbs Catalyst 2^{nd} Generation (27 milligrams, 0.04 millimoles) was dissolved in 1 mL of nitrogen-purged DCM, and added quickly to the stirring reaction mixture under nitrogen. The flask was protected from light by covering with aluminum foil. After 50 minutes, an excess of ethyl vinyl ether (200 microliters) was injected and the stirring was continued for 15 minutes. Polymer 8 was precipitated into 200 mL of a 1:1 hexane:diethyl ether mixture. Polymer 8 was isolated by filtration, dissolved in a minimal amount of DCM, and precipitated again in the same hexane:diethyl ether solution to give Polymer 8 as a purple-gray solid. The polymer was found to have a number average molecular weight (Mn) of 28,256 Daltons, as determined using gel permeation chromatography in tetrahydrofuran against polystyrene standards. ¹H NMR (400 MHz, CDCl₃) 11.46 (s, 1H), 8.42 (br, 1H), 6.09 (s, 2H), 5.79 (br, 2H), 5.0 (br, 2H), 4.45 (br, 2H), 3.62 (br, 19H), 3.35 (br, 6H), 3.34 (s, 3H), 1.85 (br, 2H), 1.48 (s, 18H).

To a 50 mL round bottom flask equipped with a stir bar Polymer 8 (200 milligrams) was added. In a separate vessel, DCM was purged with nitrogen gas for five minutes. Subsequently, 5 mL was added to the flask containing Polymer 8. The flask was sealed with a septum and purged with nitrogen gas for five minutes. The nitrogen pressure was reduced to a steady stream. Excess trifluoroacetic acid (5 mL) was added and the reaction mixture was allowed to stir for two hours. TFA was removed by rotary evaporation with DCM (3×). The polymer residue was dissolved in a minimal amount of water, filtered through a polyethersulfone (PES) syringe filter and lyophilized to yield Polymer 9 as an off-white solid which readily dissolved in water. The polymer was found to have a number average molecular weight of approximately 21,500 Daltons, as determined ¹H NMR spectroscopy, which confirmed complete removal of the boc protecting groups. ¹H NMR (400 MHz, D₂O) 5.59 (br, 2H), 5.8 (br, 2H), 4.89 (br, 2H), 4.51 (br, 2H), 3.53 (br, 23H), 3.26 (s, 3H), 3.09 (br, 2H), 1.75 (br, 2H).

The corresponding guanidinium-containing homopolymers were prepared using an analogous procedure.

Polymer Nanocapsule Preparation.

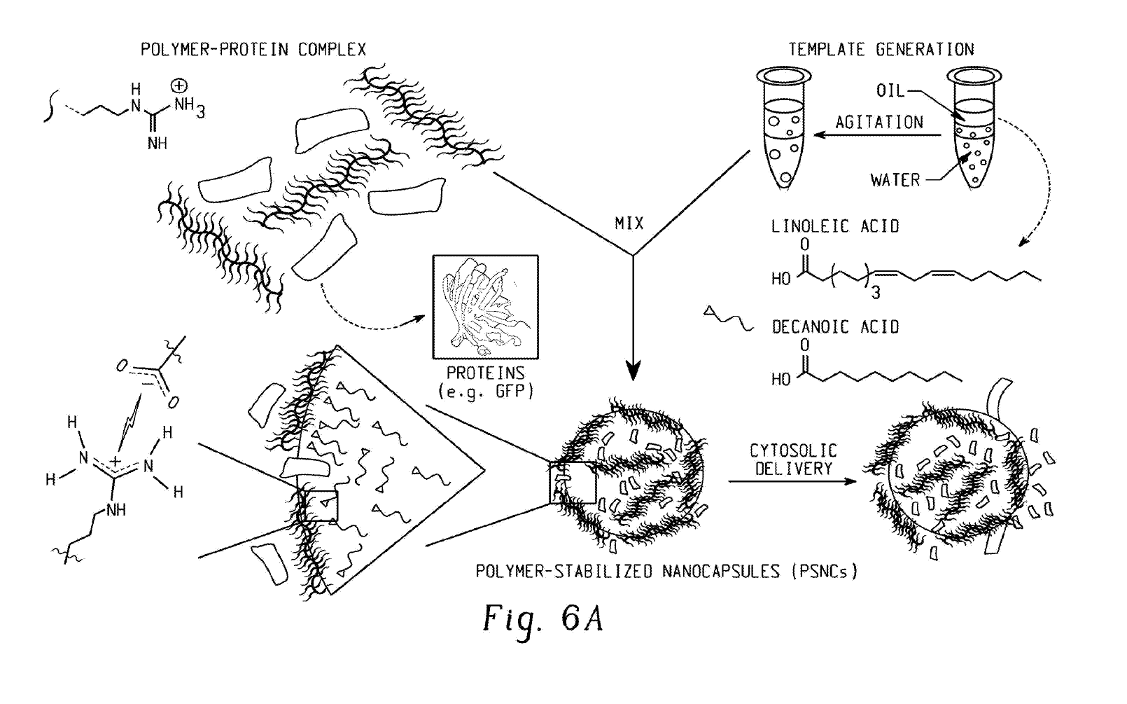

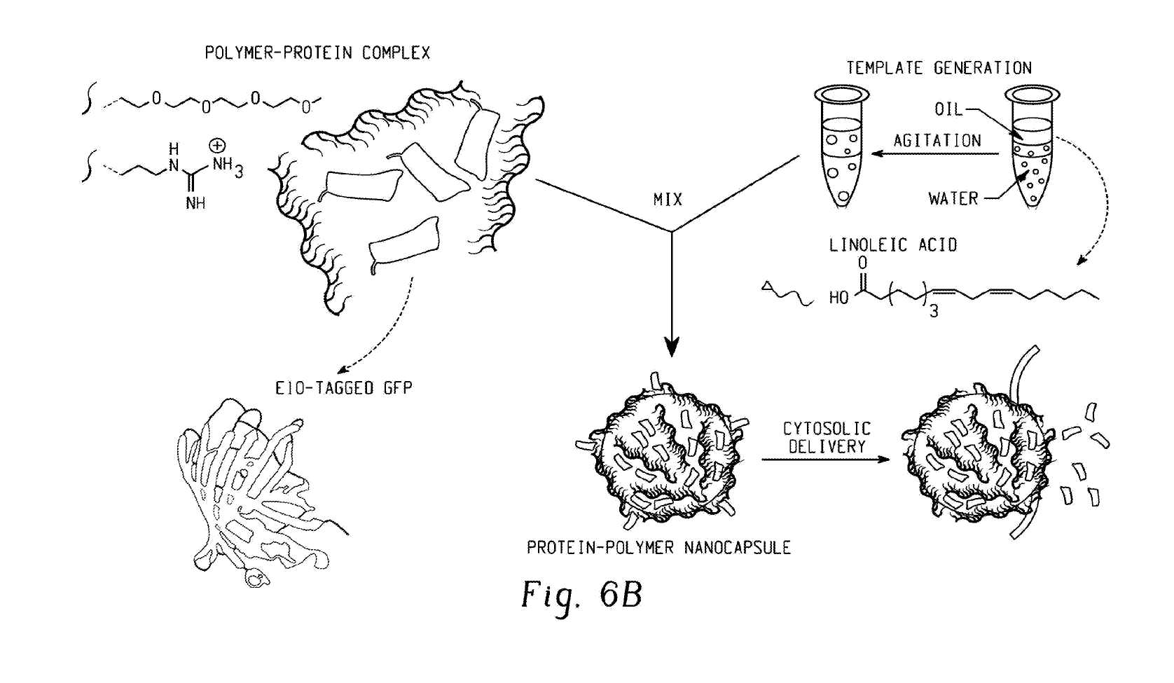

Polymer nanocapsules including the polymer 9 or the homopolymer and the protein were prepared according to the following experimental procedure, and as summarized in FIGS. 6A (homopolymer) and 6B (copolymer). Green fluorescent protein (GFP) was used as a model protein. As used herein, the term “green fluorescent protein” (GFP) refers to a protein originally isolated from the jellyfish Aequorea victoria that fluoresces green when exposed to blue light or a derivative of such a protein.

Protein-polymer nanocapsules were prepared by adding 1 microliter of linoleic acid (LA) to a 600 microliter eppendorf tube. 5 Millimolar (mM) phosphate buffer (PB, pH=7.4, 499 microliters) was added to the eppendorf tube. The tube was shaken using an amalgamator (Model: YDM-Pro) for 99 seconds on high to generate an oil template emulsion. In some examples, particularly for the preparation of nanocapsules from the guanidinium-containing homopolymers, the oil was a mixture of linoleic acid and decanoic acid in a 1:1 molar ratio.

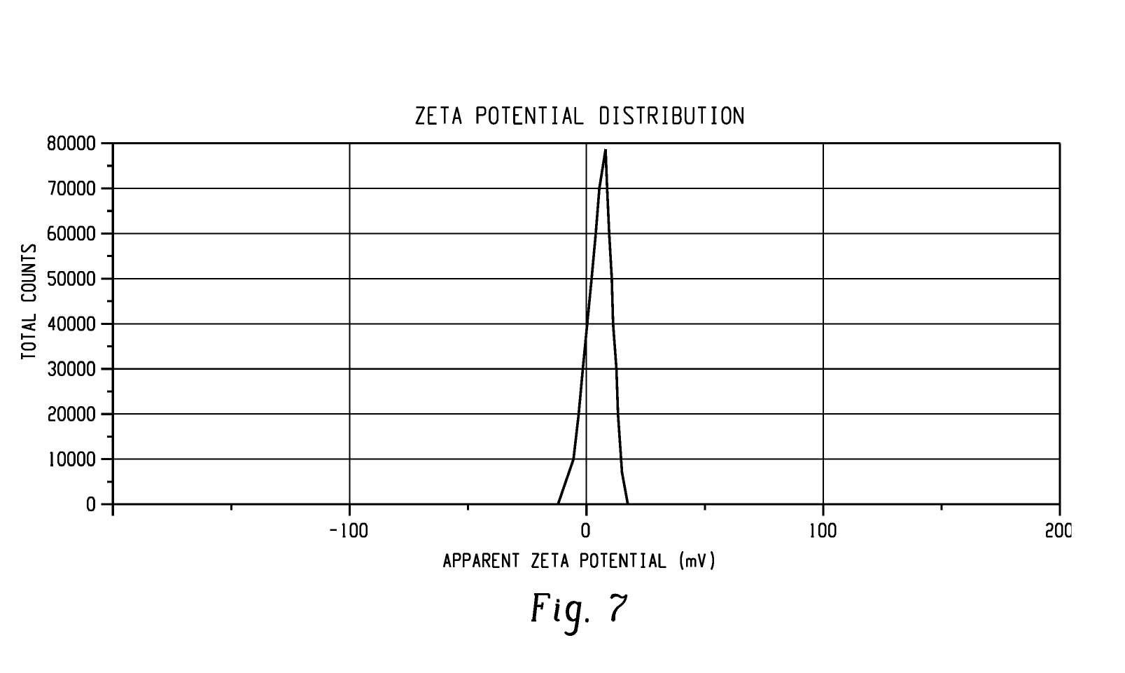

A protein-polymer complex (e.g., E-10 modified GFP) was formed by mixing E-10 modified green fluorescent protein (E-10 tagged GFP; 10 microliters of a 6 micromolar solution in pH 7.4 5 millimolar (mM) phosphate buffer) and Polymer 9 (5 microliters of a 102.3 micromolar solution in water) in phosphate buffer (30 microliters). The E-10 tag is a highly negatively charged glutamic acid tag having 10 glutamic acid units, which is believed to facilitate an electrostatic interaction between the protein and the polymer. The resulting solution was incubated for 10 minutes. Without wishing to be bound by theory, it is believed that during this incubation time, electrostatic interactions between the protein and polymer result in the formation of the protein-polymer complex. Zeta potential measurements of the nanocapsules prepared from polymer 9 indicate that the nanocapsules are slightly cationic (zeta potential of about 5 mV, as shown in FIG. 7), which is believed to facilitate attachment of the nanocapsules to cell surfaces.

The oil template emulsion (5 microliters) was then added to the protein-polymer complex solution, and mixed thoroughly (20 seconds) using a pipette.

Transmission electron microscopy (TEM) was used to confirm the formation of polymeric nanocapsules having an average diameter of less than 100 nanometers.

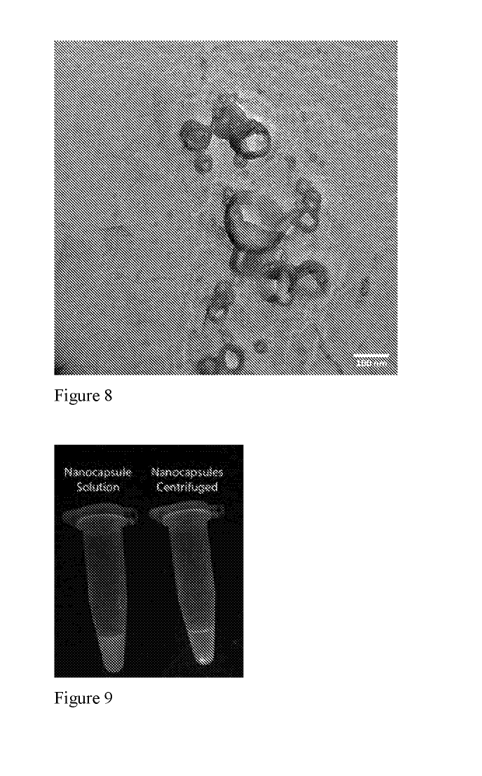

As shown in FIG. 8, polymer nanocapsules prepared from polymer 9 were imaged using TEM by casting a solution of the nanocapsules, and staining with uranyl acetate (2% aqueous solution). TEM was performed using a JEOL JEM-200FX instrument. The accelerating voltage was 200 kV. TEM showed the polymer nanocapsules formed were less than 100 nanometers in diameter. TEM also showed the nanocapsules were well dispersed, and not aggregated.

As a control experiment, a dye, Nile Red (NR), was loaded inside the nanocapsules prepared from polymer 9 and centrifuged to visually confirm capsule formation. Nile Red (0.5 milligrams) was dissolved into 100 microliters of linoleic acid. An oil template emulsion was prepared as described above using the linoleic acid/Nile Red mixture. As shown in FIG. 9, the nanocapsule solution containing Nile Red dye appeared homogenously fluorescent (left). After centrifugation of the nanocapsule solution, the fluorescence intensity of the solution is decreased, confirming that the Nile Red dye is encapsulated in the nanocapsules, which have been removed from the solution during centrifugation.

The protein-loaded polymer nanocapsules were used for cytosolic delivery of Nile Red, GFP, E-10 tagged GFP, red fluorescent protein (DsRed), siRNA, and fluorescein-tagged FIH protein (FIH-FITC). Experimental details follow.

HeLa cells (a cervical cancer cell line) were cultured in a humidified atmosphere (5% CO₂) at 37° C. and grown in Dulbecco's modified eagle's medium (DMEM, low glucose) supplemented with 10% fetal bovine serum (FBS) and 1% antibiotics (100 U/ml penicillin and 100 μg/ml streptomycin). HeLa cells were plated in either 24-well cell culture plates (60,000 cells) or confocal dishes (120,000 cells).

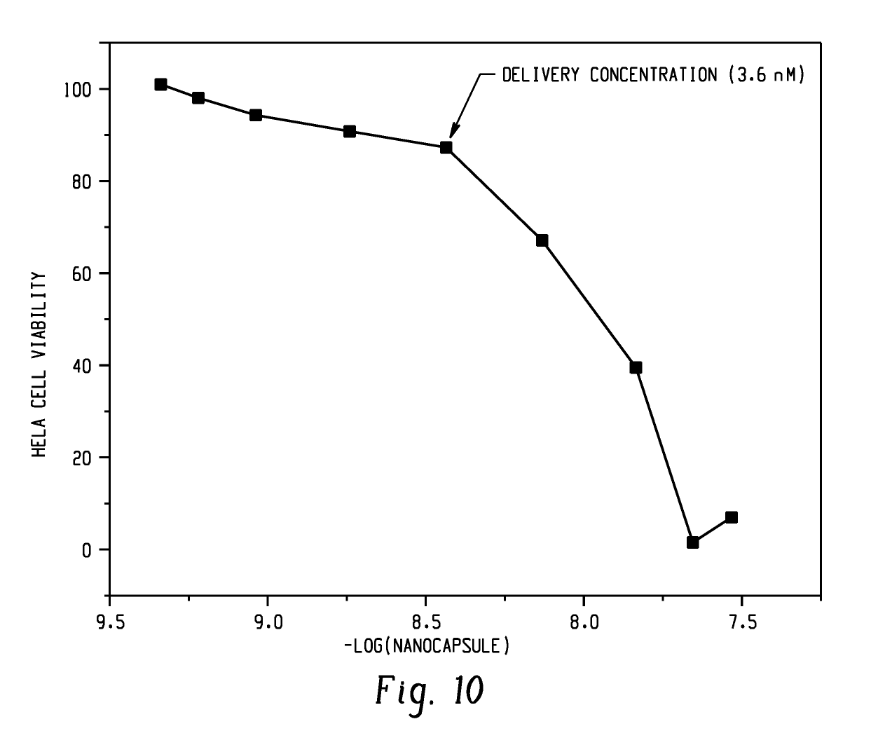

HeLa cell viability with the polymer nanocapsules was first tested, and the results are shown in FIG. 10. Polymer nanocapsules at different concentrations were incubated with HeLa cells in a 96 well plate in serum containing media for 24 hours, at which point the HeLa cells were then washed with PBS and the viability was determined using an Alamar Blue Assay. As noted in FIG. 10, the HeLa cells were greater than 80% viable after 24 hour incubation with the nanocapsules at the delivery concentration used for the following experiments (3.6 nanomolar).

Protein-polymer nanocapsules were prepared as described above. Polymer nanocapsules including DsRed and FIE protein were prepared according to the same procedure used for E-10 tagged GFP. In order to obtain sufficient confocal images, the volume amount of capsules was increased three times to obtain a nanocapsule solution having a total volume of 150 μL. The nanocapsule solution was then diluted to 500 μL using DMEM media, and incubated with HeLa cells for one hour. After one hour, the cells were washed and imaged using confocal microscopy.

The confocal microscopy results are shown in FIG. 11. As shown in FIG. 11, the E-10 tagged GFP (A and B), DsRed (C and D) proteins, and FIH-FITC (E) were successfully delivered intracellularly to the HeLa cells, confirmed by the intracellular fluorescence arising from the presence of the fluorescent proteins. FIG. 11 (F)-(J) show the corresponding bright field images of the HeLa cells. Successful delivery was achieved using nanocapsules prepared from both the homopolymer and polymer 9. Delivery of E-10 tagged GFP, DsRed, and FIH-FITC using nanocapsules prepared with the homopolymer is shown in FIGS. 11(A), 11(B), 11(C), and 11(E) (and their corresponding bright field images, 11(F), 11(G), 11(H), and 11(J)). Delivery of DsRed to the HeLa cells using nanocapsules prepared from polymer 9 is shown in FIGS. 11(D) and 11(I).

Nile Red-loaded nanocapsules were prepared as described above using polymer 9. Briefly, the nanocapsules were formed by mixing 25 μL of the Nile Red encapsulated oil template emulsion with polymer 9 using a pipette, diluted to 2 mL using DMEM media, incubated with HeLa cells for one hour, washed, and imaged using confocal microscopy.

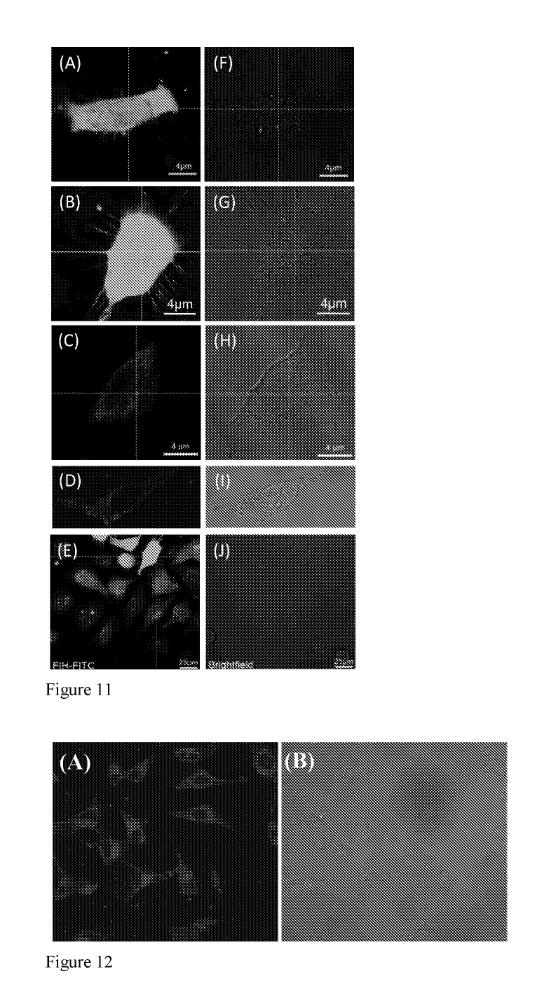

The confocal microscopy results are shown as FIG. 12. As shown in FIG. 12A, Nile Red was successfully delivered intracellularly to the HeLa cells, confirmed by the intracellular fluorescence arising from the presence of the Nile Red. FIG. 12B shows the corresponding bright field microscopy images. This demonstrates the nanocapsules of the present disclosure can be used to deliver hydrophobic moieties to cells using Nile Red as a model hydrophobe.

The nanocapsules were also tested for the ability to deliver siRNA using a cyanine (Cy-3) labeled scrambled siRNA. siRNA-polymer nanocapsules were formed using the same procedure as for the polymer-protein nanocapsules with polymer 9. The volume amount of capsules was increased five times to a total volume of 250 μL (e.g., siRNA: 12.5 of a 20 μM solution; Polymer 9: 50 μL of a 102.3 μM solution; 162.5 μL of phosphate buffer (PB); 25 μL oil template emulsion). The nanocapsule solution was diluted to 2 mL using DMEM media, and incubated with HeLa cells for one hour. The cells were washed and imaged using confocal microscopy.

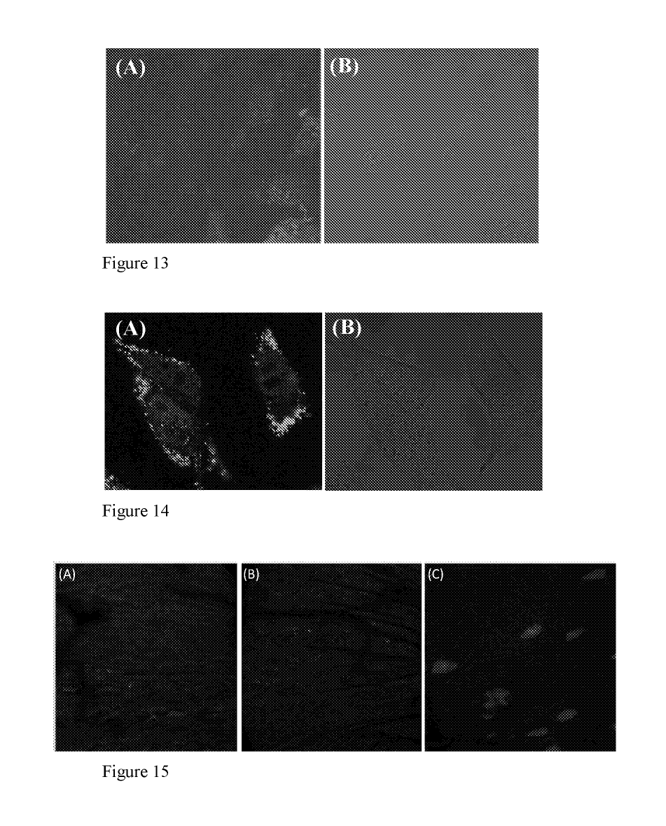

The confocal microscopy results are shown as FIG. 13. As shown in FIG. 13A, the Cy-labeled siRNA was successfully delivered intracellularly to the HeLa cells, confirmed by the intracellular fluorescence arising from the presence of the fluorescent dye. FIG. 13B shows the corresponding bright field microscopy images. This demonstrates the nanocapsules of the present disclosure can be used to deliver therapeutic agents in addition to proteins to cells.

Nanocapsules were prepared using unmodified GFP (i.e., no E10 glutamic acid tag). The nanocapsules were formed according to the same procedure used above using polymer 9. The volume amount of capsules was increased three times to a total volume of 1504, (e.g., GFP: 304, of a 6 μM solution, Polymer 9: 30 μL of a 102.3 μM solution, 75 μL PB, and 15 μL oil template emulsion), diluted to 500 μL using DMEM media. The resulting nanocapsule solution was incubated with HeLa cells for one hour, washed, and subsequently imaged using confocal microscopy.

The confocal microscopy results are shown in FIG. 14. As shown in FIG. 14A, ummodified GFP was successfully delivered intracellularly to the HeLa cells, confirmed by the intracellular fluorescence arising from the presence of the fluorescent proteins. FIG. 14B shows the corresponding bright field images of the HeLa cells after incubation with the nanocapsules.

The protein-loaded polymer nanocapsules prepared from polymer 9 were used for oral delivery of GFP to the intestinal stem cells of fruit flies. Experimental details follow.

The GFP-loaded polymer nanocapsules were prepared as described above, and orally administered to the fruit flies. The nanocapsule solution was added to a 96 well plate, and one fruit fly was added to each well. The fruit flies were allowed to feed on the nanocapsule solution for a period of 24 hours. After 24 hours, the fruit flies were sacrificed, dissected, and the intestinal stem cells with imaged using confocal microscopy as shown in FIG. 15.

FIG. 15A shows the intestinal stem cells of fruit flies that were fed sucrose (control). FIG. 15B shows the intestinal stem cells of fruit flies that were fed GFP (control). FIG. 15C shows the intestinal stem cells of fruit flies that were fed the GFP-loaded polymer nanocapsules. As can be seen from FIG. 15C, oral administration of the GFP-containing polymer nanocapsules results in successful in vivo delivery to the intestinal stem cells of fruit flies.

The invention includes at least the following embodiments.

### Embodiment 1

A nanocapsule comprising, a shell comprising a polymer and a protein, a nucleic acid, or a combination thereof, the polymer comprising repeating units of Formula (I), wherein X is independently at each occurrence —O—, —S—, —CH₂—, —(CR³R⁴)—, or

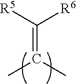

wherein R³ and R⁴ are independently at each occurrence a C₁₋₆ alkyl group and R⁵ and R⁶ are independently at each occurrence hydrogen or a C₁₋₆ alkyl group; L¹ is independently at each occurrence a divalent group that is (—CH₂-)_{z}, wherein is z is an integer from 1 to 10, a divalent a C₁₋₂₀ alkylene oxide group, or a divalent polyethylene oxide group; R¹ is independently at each occurrence hydrogen, a C₁₋₁₂ alkylene group, or a C₁₋₆—(C═O)O-alkyl group; y is 2 or 3; and a core defined by the shell, the core comprising an oil.

### Embodiment 2

The nanocapsule of embodiment 1, wherein the polymer is a copolymer further comprising repeating units of Formula (II), wherein X is independently at each occurrence —O—, —S—, —CH₂—, —(CR³R⁴)—, or

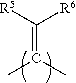

wherein R³ and R⁴ are independently at each occurrence a C₁₋₆ alkyl group and R⁵ and R⁶ are independently at each occurrence hydrogen or a C₁₋₆ alkyl group; and R² is independently at each occurrence a C₁₋₁₂ alkylene group, a C₆₋₂₀ arylene group, a C₁₋₂₀ alkylene oxide group, a polyethylene oxide group, or a zwitterionic group.

### Embodiment 3

The nanocapsule of embodiment 1 or 2, wherein the oil comprises a C₁₂₋₂₄ fatty acid.

### Embodiment 4

The nanocapsule of any of embodiments 1 to 3, wherein the oil comprises linoleic acid.

### Embodiment 5

The nanocapsule of any of embodiments 1 to 4, wherein the polymer and the protein, nucleic acid, or a combination thereof form a polymer-protein complex, a polymer-nucleic acid complex, or a combination thereof.

### Embodiment 6

The nanocapsule of any of embodiments 2 to 5, wherein the molar ratio of units of Formula (I) to units of Formula (II) is 1:2 to 2:1.

### Embodiment 7

The nanocapsule of any of embodiments 1 to 6, wherein X is —O—.

### Embodiment 8

The nanocapsule of any of embodiments 1 to 7, wherein L¹ is propylene.

### Embodiment 9

The nanocapsule of any of embodiments 1 to 8, wherein R¹ is hydrogen.

### Embodiment 10

The nanocapsule of any of embodiments 1 to 9, wherein R¹ is hydrogen and y is 3.

### Embodiment 11

The nanocapsule of any of embodiments 2 to 10, wherein R² is a C₁₋₂₀ alkylene oxide group.

### Embodiment 12

The nanocapsule of any of embodiments 2 to 11, wherein R² is a group having the structure

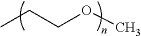

wherein n is 1, 2, 3, or 4.

### Embodiment 13

The nanocapsule of any of embodiments 1 to 12, wherein the polymer has a number average molecular weight of 10,000 to 100,000 Daltons.

### Embodiment 14

The nanocapsule of any of embodiments 1 to 13, wherein the protein has a molecular weight of 500 to 200,000 Da.

### Embodiment 15

The nanocapsule of any of embodiments 1 to 14, wherein the protein comprises a clustered regularly interspaced short palindromic repeat (CRISPR) associated protein, a caspase protein, a tyrosine recombinase enzyme, or a ribonuclease.

### Embodiment 16

The nanocapsule of any of embodiments 1 to 15, wherein the protein comprises a negatively charged group comprising a peptide group.

### Embodiment 17

The nanocapsule of any of embodiments 1 to 16, wherein the nucleic acid comprises a ribonucleic acid.

### Embodiment 18

The nanocapsule of any of embodiments 1 to 17, wherein the nanocapsule has a diameter of 1 to 100 nanometers.

### Embodiment 19

The nanocapsule of any of embodiments 1 to 18, further comprising a peptide, a nucleic acid, an oligonucleotide, a polynucleotide, a hydrophobic drug, an imaging agent, or a combination thereof.

### Embodiment 20

The nanocapsule of any of embodiments 1 to 19, wherein the shell comprises the polymer comprising repeating units of Formula (I) and the protein; each occurrence of X is —O—; each occurrence of L¹ is propylene; each occurrence of R¹ is hydrogen and y is 3; the oil comprises linoleic acid; and the nanocapsule has a diameter of 1 to 100 nanometers.

### Embodiment 21

The nanocapsule of any of embodiments 2 to 19, wherein the shell comprises the copolymer comprising repeating units of Formula (I) and (II) and the protein; each occurrence of X is —O—; each occurrence of L¹ is propylene; each occurrence of R¹ is hydrogen and y is 3; each occurrence of R² is a group having the structure

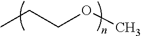

wherein n is 4; the oil comprises linoleic acid; and the nanocapsule has a diameter of 1 to 100 nanometers.

### Embodiment 22

The nanocapsule of any of embodiments 1 to 19, wherein the shell comprises the polymer comprising repeating units of Formula (I) and the nucleic acid; each occurrence of X is —O—; each occurrence of L¹ is propylene; each occurrence of R¹ is hydrogen and y is 3; the oil comprises linoleic acid; and the nanocapsule has a diameter of 1 to 100 nanometers.

### Embodiment 23

The nanocapsule of any of embodiments 2 to 19, wherein the shell comprises the copolymer comprising repeating units of Formula (I) and (II) and the nucleic acid; each occurrence of X is —O—; each occurrence of L¹ is propylene; each occurrence of R¹ is hydrogen and y is 3; each occurrence of R² is a group having the structure

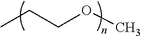

wherein n is 4; the oil comprises linoleic acid; and the nanocapsule has a diameter of 1 to 100 nanometers.

### Embodiment 24

A composition comprising a plurality of nanocapsules according to any of embodiments 1 to 23 dispersed in an aqueous solution.

### Embodiment 25

A method of preparing the nanocapsule of any of embodiments 1 to 23, the method comprising contacting a first aqueous solution comprising the polymer with a second aqueous solution comprising the protein, the nucleic acid, or combination thereof, to provide a reaction mixture comprising a polymer-protein complex, a polymer-nucleic acid complex, or a combination thereof; and contacting the reaction mixture with an emulsion comprising spherical droplets of the oil dispersed in an aqueous phase to provide the nanocapsule.

### Embodiment 26

A method of delivering a protein, a nucleic acid, or a combination thereof into a cell, the method comprising, contacting the nanocapsule of any of embodiments 1 to 23 with a cell.

### Embodiment 27

The method of embodiment 26, wherein the protein, the nucleic acid, or a combination thereof are released from the nanocapsule after contacting the nanocapsule with the cell.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

As used herein, the term “alkyl” means a branched or straight chain, saturated, monovalent hydrocarbon group, e.g., methyl, ethyl, i-propyl, and n-butyl. “Alkylene” means a straight or branched chain, saturated, divalent hydrocarbon group (e.g., methylene (—CH2-) or propylene (—(CH2)3-)). “Alkenyl” and “alkenylene” mean a monovalent or divalent, respectively, straight or branched chain hydrocarbon group having at least one carbon-carbon double bond (e.g., ethenyl (—HC═CH2) or propenylene (—HC(CH3)=CH2-). “Alkynyl” means a straight or branched chain, monovalent hydrocarbon group having at least one carbon-carbon triple bond (e.g., ethynyl). “Alkoxy” means an alkyl group linked via an oxygen (i.e., alkyl-O—), for example methoxy, ethoxy, and sec-butyloxy. “Cycloalkyl” and “cycloalkylene” mean a monovalent and divalent cyclic hydrocarbon group, respectively, of the formula —CnH2n-x and —CnH2n-2x- wherein x is the number of cyclization(s). “Aryl” means a monovalent, monocyclic or polycyclic aromatic group (e.g., phenyl or naphthyl). “Arylene” means a divalent, monocyclic or polycyclic aromatic group (e.g., phenylene or naphthylene). The prefix “halo” means a group or compound including one more halogen (F, Cl, Br, or I) substituents, which can be the same or different. The prefix “hetero” means a group or compound that includes at least one ring member that is a heteroatom (e.g., 1, 2, or 3 heteroatoms, wherein each heteroatom is independently N, O, S, or P.

“Substituted” means that the compound or group is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g, benzyl), C₇₋₁₂ alkylarylene (e.g, toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the group, including those of the substituent(s).

1. A nanocapsule comprising,
a shell comprising
a protein, a nucleic acid, or a combination thereof; and
a polymer comprising repeating units of Formula (I) 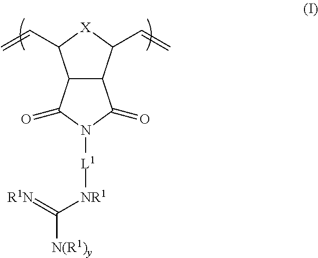
wherein
X is independently at each occurrence —O—, —S—, —CH₂—, —(CR³R⁴)—, or 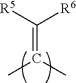
wherein R³ and R⁴ are independently at each occurrence a C₁₋₆ alkyl group and R⁵ and R⁶ are independently at each occurrence hydrogen or a C₁₋₆ alkyl group;
L¹ is independently at each occurrence a divalent group that is (—CH₂-)_{z}, wherein z is an integer from 1 to 10, a divalent a C₁₋₂₀ alkylene oxide group, or a divalent polyethylene oxide group;
R¹ is independently at each occurrence hydrogen, a C₁₋₁₂ alkylene group, or a C₁₋₆—(C═O)O-alkyl group;
y is 2 or 3;
and
a core defined by the shell, the core comprising an oil.

2. The nanocapsule of claim 1, wherein the polymer is a copolymer further comprises repeating units of Formula (II) 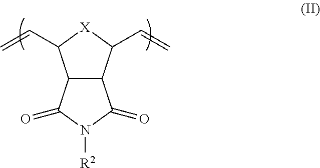
wherein
X is independently at each occurrence —O—, —S—, —CH₂—, —(CR³R⁴)—, or 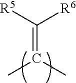
wherein R³ and R⁴ are independently at each occurrence a C₁₋₆ alkyl group and R⁵ and R⁶ are independently at each occurrence hydrogen or a C₁₋₆ alkyl group; and
R² is independently at each occurrence a C₁₋₁₂ alkylene group, a C₆₋₂₀ arylene group, a C₁₋₂₀ alkylene oxide group, a polyethylene oxide group, or a zwitterionic group.

3. The nanocapsule of claim 1, wherein the oil comprises a C₁₂₋₂₄ fatty acid.

4. The nanocapsule of claim 1, wherein the polymer and the protein, nucleic acid, or a combination thereof form a polymer-protein complex, a polymer-nucleic acid complex, or a combination thereof.

5. The nanocapsule of claim 2, wherein the molar ratio of units of Formula (I) to units of Formula (II) is 1:2 to 2:1.

6. The nanocapsule of claim 1, wherein
X is —O—;
L¹ is propylene; and
R¹ is hydrogen and y is 3.

7. The nanocapsule of claim 2, wherein R² is a C₁₋₂₀ alkylene oxide group having the structure 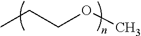
wherein n is 1, 2, 3, or 4.

8. The nanocapsule of claim 1, wherein
the polymer has a number average molecular weight of 10,000 to 100,000 Daltons; and
the protein has a molecular weight of 500 to 200,000 Daltons.

9. The nanocapsule of claim 1, wherein the protein comprises a clustered regularly interspaced short palindromic repeat (CRISPR) associated protein, a caspase protein, a tyrosine recombinase enzyme, or a ribonuclease.

10. The nanocapsule of claim 1, wherein the protein comprises a negatively charged group comprising a peptide.

11. The nanocapsule of claim 1, wherein the nucleic acid comprises a ribonucleic acid.

12. The nanocapsule of claim 1, wherein the nanocapsule has a diameter of 1 to 100 nanometers.

13. The nanocapsule of claim 1, further comprising a nucleic acid, an oligonucleotide, a polynucleotide, a hydrophobic drug, an imaging agent, or a combination thereof.

14. The nanocapsule of claim 1, wherein
the shell comprises the polymer comprising repeating units of Formula (I) and the protein;
each occurrence of X is —O—;
each occurrence of L¹ is propylene;
each occurrence of R¹ is hydrogen and y is 3;
the oil comprises linoleic acid; and
the nanocapsule has a diameter of 1 to 100 nanometers.

15. The nanocapsule of claim 2, wherein
the shell comprises the copolymer comprising repeating units of Formula (I) and (II) and the protein;
each occurrence of X is —O—;
each occurrence of L¹ is propylene;
each occurrence of R¹ is hydrogen and y is 3;
each occurrence of R² is a group having the structure 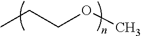
wherein n is 4;
the oil comprises linoleic acid; and
the nanocapsule has a diameter of 1 to 100 nanometers.

16. The nanocapsule of claim 1, wherein
the shell comprises the polymer comprising repeating units of Formula (I) and the nucleic acid;
each occurrence of X is —O—;
each occurrence of L¹ is propylene;
each occurrence of R¹ is hydrogen and y is 3;
the oil comprises linoleic acid; and
the nanocapsule has a diameter of 1 to 100 nanometers.

17. The nanocapsule of claim 2, wherein
the shell comprises the copolymer comprising repeating units of Formula (I) and (II) and the nucleic acid;
each occurrence of X is —O—;
each occurrence of L¹ is propylene;
each occurrence of R¹ is hydrogen and y is 3;
each occurrence of R² is a group having the structure 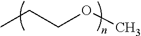
wherein n is 4;
the oil comprises linoleic acid; and
the nanocapsule has a diameter of 1 to 100 nanometers.

18. A composition comprising a plurality of nanocapsules according to claim 1 dispersed in an aqueous solution.

19. A method of preparing the nanocapsule of claim 1, the method comprising
contacting a first aqueous solution comprising the polymer with a second aqueous solution comprising the protein, the nucleic acid, or combination thereof, to provide a reaction mixture comprising a polymer-protein complex, a polymer-nucleic acid complex, or a combination thereof; and
contacting the reaction mixture with an emulsion comprising spherical droplets of the oil dispersed in an aqueous phase to provide the nanocapsule.

20. A method of delivering a protein, a nucleic acid, or a combination thereof into a cell, the method comprising,
contacting the nanocapsule of claim 1 with a cell;
wherein the protein, the nucleic acid, or combination thereof is released from the nanocapsule after contacting the nanocapsule with the cell.


SEQUENCE LISTING (3 sequences)

SEQ ID NO: 1 - protein, 7 aa, Simian virus 40
PKKKRKV

SEQ ID NO: 2 - protein, 9 aa, Homo sapiens
PAAKRVKLD

SEQ ID NO: 3 - protein, 25 aa, Caenorhabditis elegans
MSRRRKANPTKLSENAKKLAKEVEN